(19) United States
(12) Patent Application Publication
(10) Pub. No.: US 2012/0080029 A1
(43) Pub. Date: Apr. 5, 2012

(54) Discharge device for pharmaceutical media

(76) Inventors: Joachim Koerner, Uhldigen-Muehlhofen (DE); Joerg Kohnle, Vs-Schwenningen (DE); Klaus Lindenau, Radolfzell (DE)

(21) Appl. No.: 13/200,739

(22) Filed: Sep. 29, 2011

(30) Foreign Application Priority Data
Oct. 5, 2010 (DE) 102010042007.7

(51) Int. Cl. A61M 15/00; B67D 7/06

(52) U.S. Cl. 128/203.12; 222/23

(57) ABSTRACT

In order to obtain a compact construction, in the case of a discharge device (10) for pharmaceutical media comprising a housing (14, 16), which at least partly surrounds a receptacle space (12) for receiving a medium store (8), comprising a discharge opening (17) provided on the housing (14, 16) and serving for administering the medium from the medium store (8), and comprising an electronic circuit (60) designed for detecting discharge processes, it is provided that a display device (30) having curved shaping, a plurality of printed circuit boards angled relative to one another and/or a curved printed circuit board (61) are employed.

### FIELD OF APPLICATION AND PRIOR ART

The invention relates to a discharge device for pharmaceutical media, in particular for pharmaceutical liquids, comprising a housing, which at least partly surrounds a receptacle space for receiving a medium store, comprising a discharge opening provided on the housing and serving for administering the medium from the medium store, and comprising an electronic circuit designed for detecting discharge processes. In this case, the housing in discharge devices of the generic type has a convexly curved outer form at least in sections.

Discharge devices of the generic type are generally known from the prior art. They can be used by users to administer medicaments, particularly in liquid form, themselves. Discharge devices of the generic type are usually small discharge devices having a total weight of less than 200 g. These discharge devices, depending on the type, are often carried by the user. This holds true, in particular, for discharge devices for those medicaments that have to be taken regularly, such as in the case of asthma sprays, for example. The fact that discharge devices of the generic type are often carried by the user leads to the requirement that such discharge devices should be embodied as small as possible.

Discharge devices of the generic type have an electronic circuit designed for detecting discharge processes. It is thus possible, by means of such an electronic circuit, to provide the user with information about how many discharge processes have already been carried out or how many discharge processes are still possible with the medium store currently in use. The provision of such an electronic circuit designed for counting the discharge processes is not purely optional, but rather in some countries the result of a legal stipulation. Counting devices for discharge devices are known for example from WO 1991/06334 A1, WO 1996/00595 A1 and WO 2005/009325 A2. In these and other discharge devices known from the prior art, the electronic circuits required for the counting processes are realized with a rigid printed circuit board. In devices of the prior art, a display device that can usually likewise be found in such discharge devices is usually realized as an LC display having a planar surface. The display region of these LC displays is normally very small.

The design freedom in the development of discharge devices is appreciably restricted by these components mentioned, that is to say in particular the rigid and planar printed circuit board and also the rigid and planar display. The discharge devices provided therewith usually have a distinctly larger outer form than display devices without such electronic components. This is disturbing particularly in the case of discharge devices which are intended always to be carried by the user, as is the case for example with so-called MDIs (Metered-Dose Inhaler).

### PROBLEM AND SOLUTION

Therefore, the problem addressed by the invention is that of developing discharge devices of the generic type to the effect that they have a more compact construction and nevertheless provide the desired and in some instances legally required counting functionality.

This problem is solved according to the invention by virtue of the fact that the electronic circuit is connected to a display device having a display region for representing status information, wherein said display region has a curved shaping corresponding to the convexly curved outer form of the housing. As an alternative thereto or in addition, with regard to the electronic circuit, it can be provided that said circuit has a plurality of printed circuit boards which are arranged in an angled position relative to one another, which are arranged behind a curved outer wall of the housing and which are in each case oriented approximately parallel to a tangent to the outer wall. As an alternative thereto, it is also possible for the electronic circuit to have a curved printed circuit board, which is arranged behind a curved outer wall of the housing and has a curved shaping corresponding to the curved outer wall.

With regard to the display device, the central aspect of the invention resides in the fact that its display region is curved corresponding to the shaping of the housing. The outer form of the housing, which can be substantially rotationally symmetrical, for example, and is thereby curved at least in sections, thus continues in the same shaping in the display region of the display device. Consequently, at the same time it is possible to obtain a large display area without the latter per se already constraining an enlargement of the housing in its entirety.

The same similarly applies to the electronic circuit, which by definition has at least one microprocessor or a simple controller for detecting and counting discharge processes. In order to accommodate this circuit on or in the housing in a particularly space-saving manner, in accordance with a first aspect of the invention, it has a plurality of printed circuit boards which are arranged in an angled position relative to one another behind a curved outer wall of the housing. Through the use of a plurality of printed circuit boards, it is also possible to accommodate a larger number of electronic components in a space-saving manner since the printed circuit boards, on the inner side of an outer wall, can in each case be oriented approximately parallel thereto. Also advantageous by comparison therewith is the use of one curved printed circuit board, which is arranged behind a curved outer wall of the housing and is curved in a manner corresponding to the curvature of the outer wall, such that in this way, too, only a very small structural space is needed on the inner side of the outer wall of the housing. The outer wall, behind which the at least one printed circuit board of the electronic circuit is arranged, can also be formed by the display device arranged on the outer side thereof.

The curved display region of the display device and/or the curved printed circuit board can already have the respective curved shaping in the state prior to the mounting thereof, for example by virtue of the fact that they comprise in each case a, for example thermoformed, plastic substrate that is already brought to a curved position during production. As an alternative to such a rigidly curved shaping, however, the components can also be merely flexibly pliable components which are planar, if appropriate, upon a bending force being cancelled and which are fixed to the curved housing of the discharge device in the course of mounting in such a way that they likewise assume its curvature. For this purpose, in the case of the display device, for example, it is possible to adhesively bond the latter on an outer area of the housing and to constrain it into the curved shaping as a result. With regard to the printed circuit board, it can be expedient to fixedly adhesively bond it on the inner side of the outer housing and to impart to it approximately the shaping of the housing in this region as a result. The advantage of a flexibly pliable display device also resides in the fact that the latter can be used in different models of discharge devices with a mutually deviating curvature of the outer wall.

The average bending radius of the curved display device and/or of the curved printed circuit board is preferably not more than 15 cm, in particular preferably not more than 8 cm or even a maximum of 3 cm.

With regard to the display device it is regarded as preferred if said display device has a substrate, on which display segments are provided, which were applied by printing technology or coating technology.

Applying display segments on a substrate belonging to the display device constitutes a very favourable possibility for producing a display device which is advantageous with regard to a favourable production price required. The display segments can produce the form of a pixel matrix on the display device in order to be able to reproduce any desired and, if appropriate, also graphically conditioned information. It is preferred, however, if a small number of display segments, preferably fewer than 100 display segments, are used since more cost-effective production both of the display device and of the electronic circuit required for driving is thus possible. In particular, it is advantageous if one or a plurality of seven-segment displays is or are used. In addition, in order that the quantity of medium already released or the quantity of medium still contained can be registered more rapidly, a bar composed of a multiplicity of display segments can also be provided. The display segments are formed by electrophoretic or electrochromatic materials. They can consist of organic polymer compounds, for example, which are already known from the OLED field and which emit light when an electrical voltage is applied. As an alternative thereto, a display device according to E-Ink technology is also conceivable. LC displays can also be used in especially rigidly curved configurations.

The material forming the display segments is applied by way of printing technology or coating technology. The connection between the display segments and the electronic circuit for driving the segments is preferably effected by means of metallic conductors or plastic conductors which can also be applied by way of printing technology or coating technology.

In particular, a plastic substrate or a glass substrate is appropriate as the substrate. Glass substrates are usually planar in the production state. In the case of a small glass thickness, in particular in the case of a glass thickness<150 μm, however, it is possible to bend the glass without breaking to a limited extent such that the curved shaping can be obtained. However, the use of a thin plastic substrate, preferably having a thickness of between 50 μm and 500 μm, is advantageous. Said plastic substrate can be rigidly planar or else embodied in a rigidly curved fashion. In particular, a film having the material thickness mentioned can be involved. Any non-conductive plastics are appropriate as material. The use of polyester, polyamide or polyimide is particularly preferred.

Preferably, the display device has, alongside the substrate, a further plastic layer covering the display segments on the opposite side. The base substrate on which the display segments are applied and the further plastic layer are connected to one another at least in the edge region, but preferably areally, in order to avoid the ingress of liquid or air into the display device.

With regard to the curved printed circuit board, it is likewise conceivable for the latter to be embodied as a rigidly curved printed circuit board and thus as a printed circuit board already present in curved fashion prior to mounting. However, here as well a dimensionally flexible printed circuit board, in particular a printed circuit board embodied as an FPCB printed circuit board, is preferred. This printed circuit board preferably has a plastic substrate as carrier, wherein conductor tracks are provided on said plastic substrate in the form of metal films or in the form of printed conductor tracks. By virtue of the configuration of the printed circuit board as a dimensionally flexible printed circuit board, the latter can be used in the same way as dimensionally flexible display devices in the region of outer walls of the housing with different shaping. The flexible printed circuit board can therefore be inserted into a receptacle space of the housing, said receptacle space being embodied in a curved fashion, and can be supported there on an outer wall and/or an inner wall or else be fixedly adhesively bonded to an outer wall. As also in the case of the display device, preferably a thin film in the range of between 50 μm and 500 μm, which can be embodied, in particular, as a polyester film, as a polyamide film or as a polyimide film, is appropriate as the plastic substrate. A paper-like cellulose substrate can also be employed here. Preferably, films connected to one another in the edge region or areally are provided on both sides of the conductor tracks. The conductor tracks can be printed on the plastic substrate or be pressed thereon in the heated state in order to form an intimate connection to the plastic substrate. In the case of printed conductor tracks, the latter can be composed, in particular, of conductive organic polymers or of metal.

The electronic circuit has at least one microprocessor and, if appropriate, additionally a multiplexer for driving the display device. Said microprocessor and further components, such as, for example, a memory or the multiplexer, can be embodied as discrete components which are contact-connected to the conductor tracks through the plastic substrate or through a film applied opposite. In one preferred alternative configuration, it is provided that the microprocessor designed in a very simple fashion for a counting mechanism and serving for detecting discharge processes and/or for driving the display device is applied on the plastic substrate by means of printing technology. This enables a very flat design.

The discharge device according to the invention preferably furthermore has a battery for supplying the electronic circuit and/or the display device with electrical energy, wherein said battery is preferably embodied as a flat battery and is arranged behind a curved outer wall of the housing, wherein it has a curved shaping corresponding to said curved outer wall. In this way, the battery itself also occupies only a minimal structural space in the discharge device and can bear against an outer wall or be positioned in a manner almost bearing against the latter. What is preferably involved is a film battery closed on both sides by plastic films. This battery preferably has two layers applied at least in printed fashion between the plastic films, wherein the electrodes of the battery are arranged in one layer and wherein an electrolyte layer is applied in a layer arranged above or below. It is also possible to provide the two electrodes on both sides of the electrolyte layer, such that, including the two films terminating the battery towards the top and bottom, a layer composite assembly comprising at least five layers overall arises.

Furthermore, the discharge device preferably has a sensor for detecting a discharge process. Said sensor is embodied particularly preferably as a force sensor or capacitive sensor. The sensor is also preferably printed on a plastic substrate.

It is particularly advantageous if the sensor is embodied in such a way that it can detect the movement of the medium store in the receptacle space. In many discharge devices, the medium store is displaced in order thereby to bring about a discharge process. This displacement can be detected, for example, by the medium store exerting a force on a section of the sensor. This force can be detected. It is thus possible, for example, to apply, in particular to print, a strain gauge or a piezo-sensor on a plastic substrate, which, in the event of deformation of the plastic substrate, brings about a change in voltage or change in resistance that can be measured by means of a microprocessor. Said strain gauge or said piezo-sensor is preferably likewise arranged between two layers of plastic film. A capacitive sensor as an alternative thereto can likewise be applied by printing technology, as is already customary in the case of capacitive screens of present-day mobile telephones. Said sensor makes it possible to detect a movement of the medium store moved relative to the sensor in the receptacle space.

In one configuration, the housing of the discharge device has an outwardly facing outer shell and an inner shell facing the receptacle space, which together delimit an intervening electronics space, wherein the display device and/or the at least one printed circuit board are/is arranged in said electronics space. Said electronics space, which can have a very small clear width of, for example, less than 3 mm, has the curved shaping of the housing in this region. The use of the above-described components (display device, electronic circuit, sensor, battery) also permits this small structural space to be sufficient for arrangement purposes. What is preferably involved is a ring-shaped electronics space that surrounds the receptacle space in a manner separated by the inner shell.

In a configuration as an alternative thereto, the housing merely has an outwardly facing outer shell, on the inner side of which the at least one printed circuit board or the flat battery is provided, wherein no further walls of the housing are provided between the printed circuit board and the receptacle space for receiving the medium store. Consequently, in the case of such a configuration, an outer wall of the medium store lies directly opposite the inner side of the printed circuit board. A particularly slender design of the discharge device in the region of the printed circuit board or the battery can be obtained as a result.

As an alternative to the arrangement of at least the printed circuit board within the housing, it is also possible for a flexible structural unit comprising the discharge device, the electronic circuit, the sensor and the battery to be applied on the outer side on an outer wall of the housing. This makes it possible to design a discharge device according to the invention largely structurally identically relative to a hitherto conventional discharge device without electronic components with regard to the housing. The preferably intrinsically flexible composite assembly composed of the components mentioned is merely applied on the outer side on the discharge device according to the invention. This composite assembly can be fixedly adhesively bonded, in particular, for this purpose, wherein it is particularly advantageous if the composite assembly has on an inner side a self-adhesive area by means of which fitting to the housing of the discharge device is possible. Since, according to the current prior art of the respective electronic components, it is difficult to undershoot a thickness of approximately 2 to 3 mm for said composite assembly, a depression is preferably provided on the outer wall of the housing, into which depression the composite assembly is inserted, such that it is not at particular risk of damage by virtue of an exposed arrangement.

The display device and/or the outer wall, behind which the at least one flexible printed circuit board is arranged, are/is preferably provided in the region of a cylindrical housing section, wherein the cross-sectional form of said cylindrical housing section preferably forms a circular form. Such a cylindrical housing section, in particular a circular-cylindrical housing section, is found in many discharge devices which are constructed in a rotationally symmetrical fashion at least in sections. Fitting the display device and/or the printed circuit board in such a cylindrical section has the effect that flexure has to be effected in each case only in one bending plane, which facilitates the producibility of such a printed circuit board and/or such a display device. As an alternative to a circular-cylindrical form, however, it can also be advantageous if a shaping deviating therefrom is present in the housing section on which the display device or the printed circuit board is arranged. In particular, it is advantageous if the display device and/or the printed circuit board are/is provided in the region of an outer wall whose radius of curvature is greater than the maximum half-diameter of the housing in this housing section. This can be given for example by an elliptical cross section of the housing in this housing section. A partial flattening of the cross section on that side at which the display device and/or the printed circuit board are/is intended to be provided is also possible. This configuration of the housing section that deviates from the circular-cylindrical form makes it possible to avoid small bending radii with regard to the display device and/or the printed circuit board.

As set out above, all four essential components of the electronics of a discharge device according to the invention, that is to say the display device, the electronic circuit with microprocessor, the sensor for detecting a discharge process and the battery, are possible on the basis of a flexibly pliable substrate on which the corresponding components can be applied at least partly by printing technology. A considerable cost advantage during production arises if at least two of these components share a common and integral plastic substrate. Thus, in particular, the sensor can be applied on the small plastic substrate as the electronic circuit or the conductor tracks of the electronic circuit of components arranged in discrete fashion thereon such as a microprocessor. Moreover, these two components or one of them can be applied on the same plastic substrate which simultaneously serves for applying the electrochromatic or electrophoretic materials for forming display segments. It is particularly advantageous if at least two of the components having a common substrate also have a common covering film lying opposite the substrate.

It is particularly advantageous if at least three of the four components mentioned are arranged one behind another along a flexibly deformable component strand, wherein said component strand is arranged in a manner following the shaping of the cylindrical housing section and preferably in a manner distributed circumferentially over a circumference of the cylindrical housing section in and/or on the housing. In this context, a component strand is understood to mean an assemblage of at least three of the components mentioned, between which a dimensionally flexible section is respectively provided, wherein the at least two dimensionally flexible connecting sections between the components are pliable in a common plane. Thus, the strand can be arranged in a simple manner and in a very space-saving manner in and/or on a discharge device. In this case, it is advantageous if said component strand, which is provided in the region of the housing wall, surrounds the enclosed receptacle space by at least 180°, preferably by 270°. This achieves an ideal space utilization since the component strand can be made very thin on account of its large circumferential length.

As an alternative to a component strand arranged along the circumference, it is also possible for at least two of the components mentioned each to have a curved shaping and, in a manner lying adjacent to one another, to constitute the layers of a common layer composite assembly. Such a layer composite assembly thus has a plurality of components in a radial direction of the cylindrical housing section. Thus, by way of example, a flat battery of the above-described design can be arranged at the inner position. It is adjoined on the outer side by the electronic circuit with the, in particular flexible, printed circuit board. On the latter, in turn, the likewise curved display device is provided on the outer side. A singularly or multiply folded component strand with these components can be involved in this case. It is also possible for the components to be constructed in a layered fashion one on top of another and to be arranged in a layered fashion one on top of another with insulation in each case by a plastic film or by some other substrate.

The configuration according to the invention can find application in all conventional types of generic discharge devices, thus for example in layers having an integrated pump or in squeeze bottles. Particular preference is given, however, to application in those discharge devices which are embodied as an inhaler having an approximately cylindrical main section, at the end of which is situated a mouthpiece extending radially away from the main section and having the discharge opening. In this case, the main section forms the cylindrical housing section for arrangement of the display device and/or of the at least one printed circuit board. Such a construction is known from so-called MDIs (Metered-Dose Inhaler). The regulatory requirements for arrangement of a counting device are comparatively high precisely in the case of this type of discharge devices. At the same time, these are also the discharge devices which are usually carried by the user and which should therefore be made as small as possible. The use of printed circuit boards arranged according to the invention, of a printed circuit board embodied in a curved fashion according to the invention and/or of a display device curved according to the invention is therefore particularly advantageous precisely with regard to these discharge devices.

### BRIEF DESCRIPTION OF THE DRAWINGS

Further advantages and aspects of the invention emerge not only from the claims but also from the following description of preferred exemplary embodiments of the invention, which are explained below with reference to the figures, in which:

FIG. 1 shows a discharge device embodied as an MDI with and without an inserted medium container,

FIG. 2 shows the electronic components of the discharge device from FIG. 1 in a premounted state,

FIG. 3 shows the discharge device from FIG. 1 in a partly sectional view,

FIG. 4 shows a cross section through the discharge device from FIG. 1,

FIGS. 5a to 5e cross sections through alternatively constructed discharge devices, and

FIGS. 6a and 6b show alternative configurations of a discharge device according to the invention.

### DETAILED DESCRIPTION OF THE EXEMPLARY EMBODIMENTS

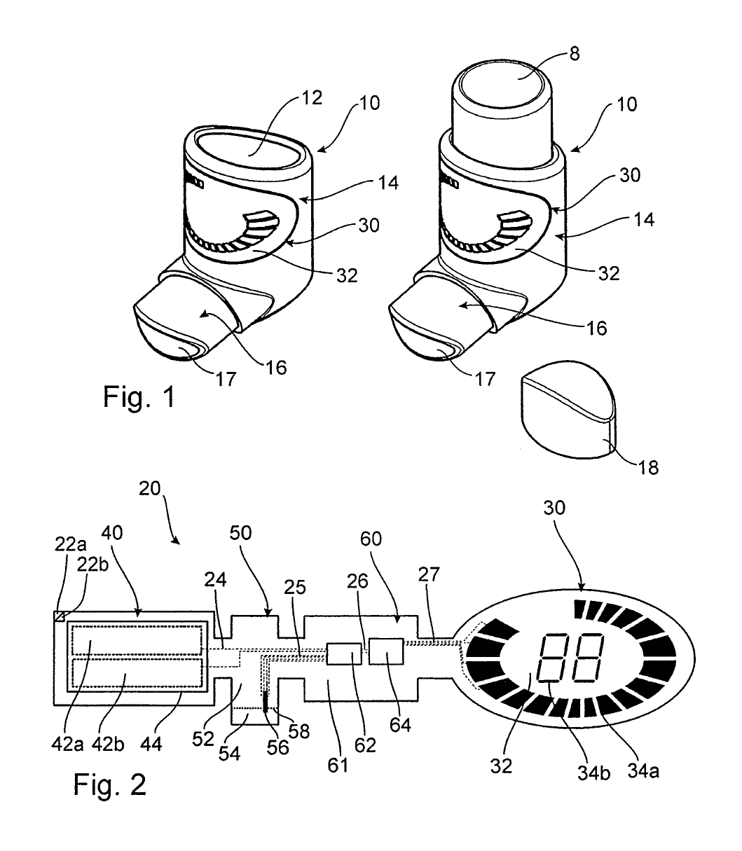

FIG. 1 shows a discharge device 10 according to the invention, wherein a medium store 8 has already been inserted in the right-hand illustration. The left-hand illustration shows the discharge device 10 without said medium store 8, such that the receptacle space 12 which is open on one side and is provided therefor can be discerned. The discharge device 10 has a housing having a cylindrical housing section 14, which surrounds said receptacle space 12, and also having a mouthpiece 16 adjoining the latter at the lower end and extending radially outwards from the housing section 14 and having a discharge opening 17, which can be closed by means of a cap 18 when not in use.

The shaping of the discharge device 10 substantially corresponds approximately to conventional MDIs (Metered Dose Inhaler). In a departure from conventional designs, however, the discharge device 10 has a display device 30 having a display region 32, which is not embodied in planar fashion, but rather is embodied in a curved fashion following the outer area of the cylindrical main section 14 of the housing and covers a comparatively large area of that side of the main section 14 which faces the user in use.

FIGS. 2 to 4 illustrate the construction of the discharge device 10. FIG. 2 shows an electronic assembly 20 used in the discharge device 10. Said electronic assembly is embodied as an elongated strand delimited over its entire length on the top side and underside by flexible plastic films 22a, 22b composed of polyamide. The two-layered nature of the films is illustrated in the top left corner for clarification. The strand-shaped assembly 20 has a total of 4 subassemblies from left to right with reference to FIG. 2. A flat battery 40 is provided on the left-hand side. In the region of said flat battery 40, two electrodes 42a, 42b are provided on the lower film 22a relative to FIG. 2. An electrolyte 44 of the battery 40 extends over said electrodes 42a, 42b, which electrolyte, in the same way as the electrodes 42a, 42b, can be embodied differently in each case depending on the type of battery. The two layers formed by the electrodes 42a, 42b, on the one hand, and the electrolytes 44 on the other hand, are terminated approximately circumferentially towards the outside by the polyamide films 22a, 22b. Both the electrodes 42a, 42b and the electrolyte 44 were applied on the lower polyamide film 22a by means of printing technology before the upper covering film 22b was fitted on the top side. The electrodes are connected to a microprocessor 62 via conductor tracks 24 likewise applied by printing technology. This will be explained hereinafter. On the right-hand side, the battery 40 is adjoined by a sensor 50. Said sensor 50 is formed by a parallel supporting section 52, which is planar with respect to the battery in the premounted state in FIG. 2, and a sensor section 54 adjoining said supporting section, wherein the supporting section 52 and the sensor section 54 are in turn embodied as a two-layered composite film assembly composed of the polyamide films 22a, 22b. In a manner that will be explained below, the sensor section 54 is angled by approximately 30° relative to the supporting section 52 in a rest state. In a translation region 58 between the sensor section 52 and the supporting section 54, a strain gauge 56 is provided, which is likewise connected to the microprocessor 62 via conductor tracks 25 likewise applied on the lower polyamide film 22a by printing technology. Said strain gauge 56 is arranged in such a way that, depending on the angle between the supporting section 52 and the sensor section 54, it constitutes a variable resistance, such that a change in this angle is detectable for the microprocessor 62. As an alternative to the strain gauge 56, by way of example, a piezo-sensor can also be provided between the polyamide films 22a, 22b, said piezo-sensor making it possible to detect flexure in the transition region 58 by outputting a deformation-dependent voltage, wherein the advantage relative to a strain gauge resides, inter alia, in obviating the need for a bridge circuit. On the right-hand side, the sensor 50 is adjoined by an electronic control unit 60. Said electronic control unit comprises the microprocessor 62 already mentioned, and also a multiplexer 64 connected to the latter via conductor tracks 25. The microprocessor 62 and the multiplexer 64, which are embodied as conventionally embodied silicon-based ICs, are placed onto a flexible printed circuit board 61 formed by the films 22a, 22b and the intervening conductor tracks 24, 25, 26, 27 applied by printing technology.

The multiplexer 64 is connected to the display device 30 via the conductor tracks 27, only some of which are illustrated by way of example. Said display device 30, like the abovementioned subassemblies 40, 50, 60, is delimited at the top side and underside by the films 22a, 22b. Display segments 34a, 34b are provided in between, said display segments in the present case being arranged in the form of a partially circular filling level bar 34a and two seven-segment displays 34b. The 34 display segments in total are connected to the multiplexer 64 by means of individual conductor tracks 27, such that they can be individually activated and deactivated by the microprocessor 62 in conjunction with the multiplexer 64.

The assembly 20 illustrated is highly advantageous in terms of production engineering. Apart from the microprocessor 62 and the multiplexer 64, only two films 22a, 22b are provided, between which various functional components which can be applied by printing technology are arranged, which together afford a complete electronic unit for the discharge device 10.

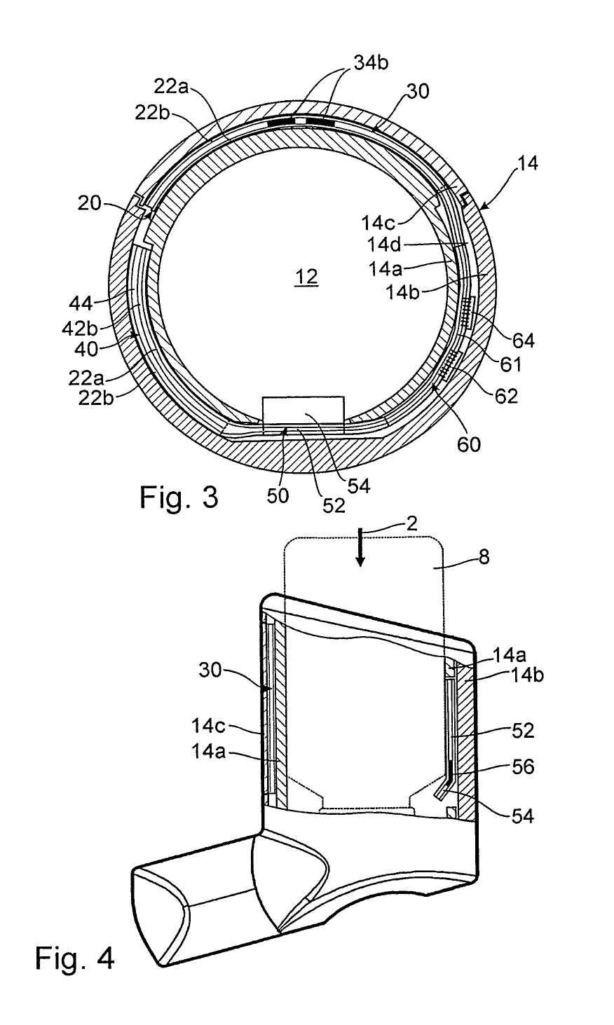

FIGS. 3 and 4 explain how the assembly 20 is arranged in the discharge device 10. Referring to FIG. 3, which shows a sectional illustration through the cylindrical main section 14 of the discharge device, it can be discerned that the rigid plastic housing of the discharge device 10 in this region substantially consists of an inner wall 14a and an outer wall 14b. The outer wall 14b has a cutout in the region of the display device 30, a transparent viewing window 14c being inserted into said cutout. Between the inner wall 14a, which directly surrounds the receptacle space 12 for receiving the medium store 8, and the outer wall 14b or the viewing window 14c, a ring-shaped receptacle space 14d is provided, in which the assembly 20 is arranged in such a way that it surrounds the receptacle space 12 approximately fully circumferentially. In this case, the display device 30 is provided directly behind the viewing window 10. In the clockwise direction, the display device 30 is adjoined by the electronic control unit subassembly 60, wherein the integrated circuits forming the microprocessor 62 and the multiplexer 64, on account of their flat design, fit into the receptacle space 14d without difficulty. Further in the clockwise direction, the electronic control unit subassembly is adjoined by the sensor subassembly 50, wherein, in the manner illustrated in FIG. 3 and in particular also in FIG. 4, it can be discerned that the sensor section 54 projects through a window in the inner wall 14a into the receptacle space 11 for the medium store 12. The sensor subassembly 50 in turn is adjoined by the flat battery 40.

As is readily apparent from FIG. 3, all the subassemblies 30, 40, 50, 60 of the component strand 20 can readily be accommodated in the receptacle space 14d by virtue of their pliability and the pliability of the tapered connecting sections located between them. The external dimensions of the discharge device 10 therefore scarcely deviate from the external dimensions of an MDI embodied without counting electronics.

During operation, as explained with reference to FIG. 4, a discharge process is brought about by the medium store 8 being pressed more deeply into the receptacle space 12 in the direction of the arrow 2. This gives rise to a deflection of the sensor section 54 relative to the supporting section 52, as a result of which the strain gauge 56 arranged in the transition region changes with regard to its resistance value. In the case where a piezo-sensor is used, the deflection would lead to the generation of a measurable voltage. This can be detected by the microprocessor 62, such that a counter available therein for discharge processes is incremented by one on account of this detected deflection. At the same time, updating of the representation on the display device 30 is also performed by the microprocessor 62 together with the multiplexer 64.

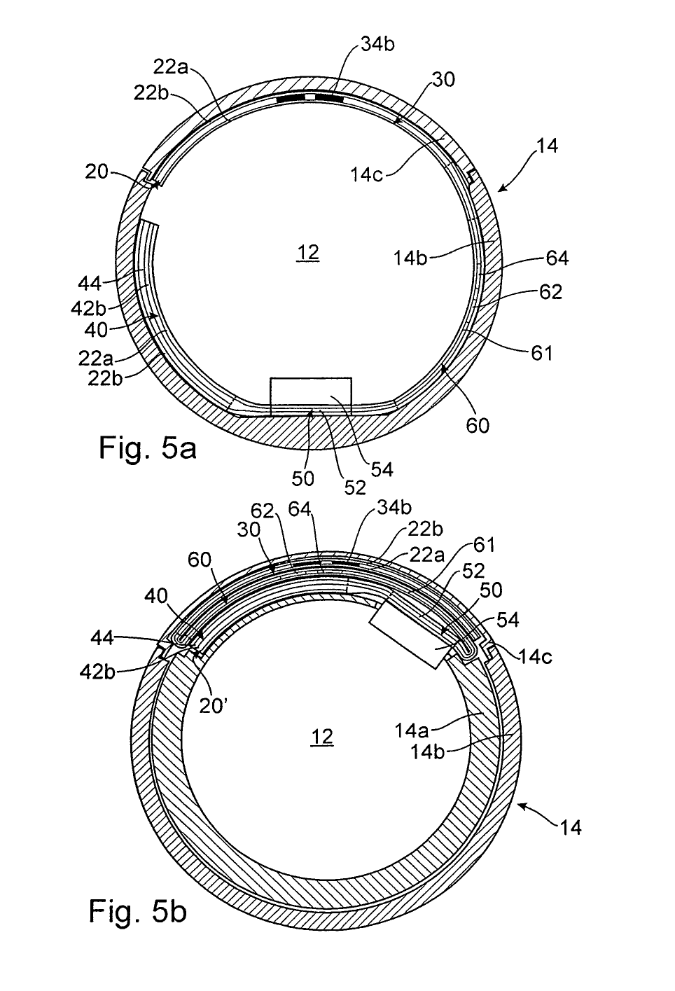

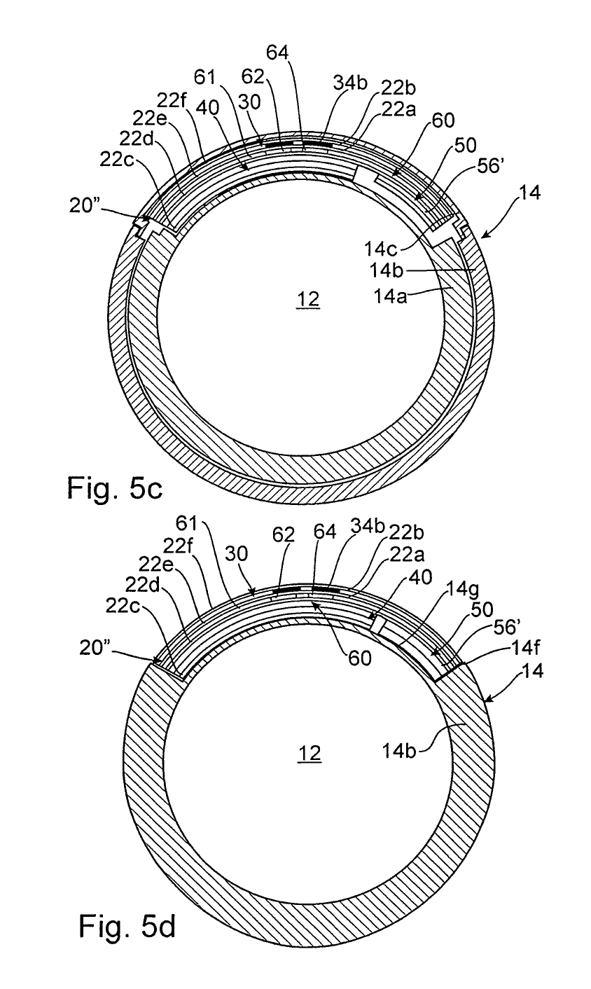

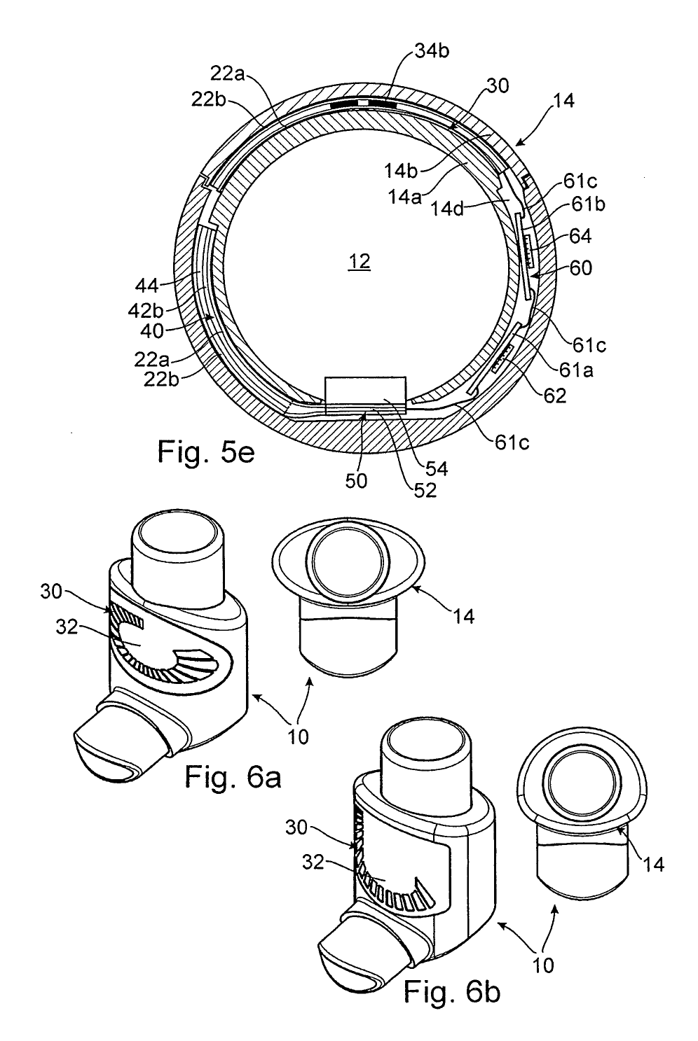

FIGS. 5a to 5e show, in a cross-sectional illustration comparable to FIG. 3, alternative configurations of the discharge device 10. Only the crucial differences are explained below. In FIGS. 5a to 5d, the microprocessor 62 and the multiplexer 64 are embodied as components printed directly between the film layers. This configuration is optional. In the examples mentioned, conventional discrete components 62, 64, as in the example in FIGS. 1 to 4, could equally be employed as well.

The configuration in FIG. 5a largely corresponds to the configuration in FIGS. 2 to 4. However, an essential difference is that the inner wall 14a is omitted in this configuration. In order to compensate for said inner wall, the subassemblies 30, 40, 50, 60, in a manner not illustrated in more specific detail, are at least partly fixed to the inner side of the outer wall 14b by means of an adhesive-bonding connection. It can easily be discerned that this design results in an even more compact construction or affords the possibility of using medium stores having a larger diameter with unchanged external dimensions in the discharge device.

A component strand 20′ is likewise employed in the configuration in accordance with FIG. 5b, said component strand substantially corresponding to the component strand 20 in FIG. 3. However, this is more particularly folded, thus resulting in a layer composite assembly arranged completely in the region of the viewing window 14c. The window for the sensor 50 on the housing inner wall 14a is therefore likewise arranged in a manner oriented towards the viewing window 14c.

FIG. 5c shows a variation in this respect. In this variation, the subassemblies 30, 40, 50, 60 are produced directly in the form of a layer composite assembly 20″, such that no bending edges arise. Instead, the respective active components of the subassemblies which are insulated from one another or from the surroundings by film layers 22c, 22d, 22e, 22f are connected to one another by means of perforations (not illustrated) in the film layers. A further difference with respect to the embodiment in FIG. 5b is that the sensor subassembly 50 in the present case has a capacitance sensor 56′ applied by printing technology. Said capacitance sensor is suitable for experiencing a change in capacitance if the medium store 8 is moved in the receptacle space 12. Since there is no need for direct touching contact between the medium store 8 and the sensor subassembly 50, in this configuration the inner wall 14a is circumferentially closed and, in a departure from the configuration in FIGS. 3 and 5b, has no window provided therefor.

In the case of the configuration in accordance with FIG. 5d, the basic construction of the assembly 20″ is comparable to that in FIG. 5c. The special feature is that only an outer wall 14b is provided which has a depression 14f over an angular range of approximately 135°, said assembly 20″ being inserted into said depression. In this case, the connection of the outer wall 14c to the assembly 20″ is preferably effected by means of an adhesive layer 14g.

The configuration in FIG. 5e largely corresponds to the configuration in FIG. 3. The special feature here is merely that, instead of a flexibly deformable printed circuit board, conventional intrinsically rigid printed circuit boards 61a, 61b are used, on which the processor 62 and the multiplexer 64 are respectively arranged. These rigid printed circuit boards 61a, 61b are connected to one another and also to the sensor subassembly 50 and the display device 30, respectively, by means of flat cables 61c (only indicated schematically). It is evident that the printed circuit boards 61a, 61b are arranged in a manner angled relative to one another in the receptacle space 14d. Thus, even when using conventional printed circuit board technology, it is possible to manage with the available space between the inner wall 14a and the outer wall 14b.

FIGS. 6a and 6b show alternative outer forms of a discharge device 10. An oval cross section of the main section 14 is provided in the case of the illustration in FIG. 6a. A cross section flattened on one side is provided in the case of the configuration in accordance with FIG. 6b. What is thereby achieved is that the bending radius in the region of the display device 20 can be greater, which can entail advantages in terms of production technology or mounting technology and, in particular, cost savings.

1. Discharge device (10) for pharmaceutical media comprising
a housing (14, 16), which at least partly surrounds a receptacle space (12) for receiving a medium store (8),
a discharge opening (17) provided on the housing (14, 16) and serving for administering the medium from the medium store (8), and
an electronic circuit (60) designed for detecting discharge processes,
wherein
the housing (14, 16) has a convexly curved outer form at least in sections,
characterized in that
the electronic circuit (60) is connected to a display device (30) having a display region (32) for representing status information, wherein the display region (32) has a curved shaping corresponding to the convexly curved outer form of the housing (14, 16), and/or
the electronic circuit (60) has a plurality of printed circuit boards (61a, 61b) which are arranged in an angled position relative to one another, which are arranged behind a curved outer wall (14b) of the housing (14, 16) and which are in each case oriented approximately parallel to said outer wall (14b), or
the electronic circuit (60) has a curved printed circuit board (61), which is arranged behind a curved outer wall (14b) of the housing (14, 16) and has a curved shaping corresponding to the curved outer wall (14b).

2. Discharge device,
characterized in that
the display device (30) has a substrate (22a), on which display segments (34a, 34b) are provided, which were applied by printing technology or coating technology.

3. Discharge device,
characterized in that
the substrate (22a) is embodied as a plastic substrate (22a) or as a glass substrate.

4. Discharge device according to claim 1,
characterized in that
the at least one curved printed circuit board (61) is embodied as a flexible printed circuit board (61), in particular as an FPCB printed circuit board, and has a plastic substrate (22a) as a carrier, wherein conductor tracks (24, 25, 26, 27) are provided on said plastic substrate (22a) in the form of metal films or in the form of printed conductor tracks (24, 25, 26, 27).

5. Discharge device according to claim 4,
characterized in that
a microprocessor (62) for detecting discharge processes and/or for driving the display device (30) is applied on the plastic substrate by means of printing technology.

6. Discharge device according to claim 1,
characterized by
a battery (40) for supplying the electronic circuit (60) and/or the display device (30) with electrical energy, wherein said battery is embodied as a flat battery and is arranged behind a curved outer wall (14b) of the housing (14, 16), wherein it has a curved shaping corresponding to the curved outer wall.

7. Discharge device according to claim 1,
characterized by
a sensor (50) for detecting a discharge process, wherein the sensor (50) is preferably embodied as a force sensor or as a capacitive sensor and wherein the sensor is preferably printed on a plastic substrate (22a).

8. Discharge device according to claim 1,
characterized in that
the housing (14, 16) has an outwardly facing outer shell (14b) and an inner shell (14a) facing the receptacle space (12), which together delimit an intervening electronics space (14d), wherein the display device (30) and/or the at least one printed circuit board (61; 61a, 61b) are/is arranged in said electronics space (14d).

9. Discharge device according to claim 1,
characterized in that
the housing has an outwardly facing outer shell (14b), on the inner side of which the at least one printed circuit board (61) or the battery (40) is provided, wherein no further walls of the housing (14, 16) are provided between the printed circuit board (61) or the battery (40), on the one hand, and the receptacle space (12) for receiving the medium store (8).

10. Discharge device according to claim 1,
characterized in that
a dimensionally flexible structural unit (20′) comprising the display device (30), the electronic circuit (60), the sensor (50) and the battery (40) is applied on the outer side on an outer wall (14b) of the housing, wherein the structural unit (20′) is preferably fixedly adhesively bonded to the outer wall.

11. Discharge device according to claim 1,
characterized in that
the display device (30) and/or the outer wall (14b, 14c), behind which the at least one printed circuit board (61; 61a, 61b) is arranged, are/is provided in the region of a cylindrical housing section (14), wherein the cross-sectional form of said cylindrical housing section (14) preferably forms a circular form.

12. Discharge device according to claim 1,
characterized in that
at least two of the four components from the group comprising the display device (30), the electronic circuit (60), a sensor (50) for detecting a discharge process and a battery (40) are applied on a common and integral plastic substrate (22a).

13. Discharge device according to claim 1,
characterized in that
at least three of the four components from the group comprising the display device (30), the electronic circuit (60), a sensor (50) for detecting a discharge process and a battery (40) are arranged one behind another along a deformable component strand (20), wherein said component strand (20) is arranged in the housing in a manner following the shaping of the cylindrical housing section.

14. Discharge device according to claim 1,
characterized in that
at least two of the four components from the group comprising the display device (30), the electronic circuit (60), a sensor (50) for detecting a discharge process and a battery (40) in each case have a curved shaping and constitute adjacent layers of a layer composite assembly (20′; 20″).

15. Discharge device according to claim 1,
characterized in that
the discharge device (10) is embodied as an inhaler (10) having an approximately cylindrical main section (14), wherein a mouthpiece (16) extending radially outward from main section (14) and having a discharge opening (17) is provided at one end of the main section (14), and wherein the main section (14) forms the cylindrical housing section (14) for the arrangement of the display device (30) and/or the at least one printed circuit board (61; 61a, 61b).
